(19) United States
(12) Patent Application Publication
(10) Pub. No.: US 2018/0280592 A1
(43) Pub. Date: Oct. 4, 2018

(54) Manually Actuated Suction Device For Aspiration Of Body Fluids, Suction Attachment For Such A Suction Device, And Kit For Aspiration Of Body Fluids Comprising A Suction Device And A Suction Attachment

(71) Applicant: MEDICPART PLUS GMBH, Berlin (DE)

(72) Inventor: Walter Lisec, Berlin (DE)

(21) Appl. No.: 15/765,315

(22) PCT Filed: Oct. 12, 2016
(86) PCT No.: PCT/EP2016/074419

§ 371 Date: Apr. 2, 2018

(30) Foreign Application Priority Data
Oct. 13, 2015 (DE) 10 2015 219 775.1

(51) Int. Cl. A61M 1/00

(52) U.S. Cl. CPC A61M 1/0003; A61M 1/0009; A61M 2205/7536; A61M 1/0068; A61M 2205/073; A61M 1/0049

(57) ABSTRACT

The present invention relates to a manually actuated suction device (1) for aspiration of body fluids, for example pus from pimples, comprising a barrel (2) with a negative-pressure chamber (3), which has a suction opening (4), a piston (5), which is displaceable in the barrel (2) and is received with play, a piston crown (6) closing the negative-pressure chamber (3) of the barrel (2) in a substantially airtight manner, and an actuation device (9) which is transferrable from a starting position (I) along an actuation direction (B) to an actuation position (II) and by which the piston (5) and the barrel (2) are displaceable relative to each other in stroke direction (H). The invention further relates to a suction attachment (50) for a suction device, comprising an attachment body (54) with a fastening portion (51) for airtight connection to a suction device (1), a suction opening (55) and a fluid line (56) from the fastening portion (51) to the suction opening (55), and a kit comprising the suction device according to the invention and at least one suction attachment according to the invention. In order to make available a manually actuated suction device for aspiration of body fluids that works reliably, has a compact structure and is easy to handle, provision is made according to the invention that the negative-pressure chamber (3) is arranged in stroke direction (H) between the piston crown (6) and a handling surface (12) of the actuation device (9). For this purpose, a liquid sorbent (57) is arranged in the fluid line (56) of the suction attachment (50) according to the invention.

The present invention relates to a manually actuated suction device for aspiration of body fluids such as pus from pimples, blackhead or nasal secretion. The suction device comprises a barrel with a negative-pressure chamber, which has an aspiration opening, a piston, which is displaceable in the barrel and received with play, a piston crown closing the negative-pressure chamber of the barrel in a substantially airtight manner, and an actuation device which is transferrable from a starting position along an actuation direction into an actuation position and by which the piston and the barrel are displaceable relative to each other in stroke direction.

The present invention further relates to a suction attachment for a suction device for aspiration of body fluids, such as pus from pimples, comprising an attachment body with a fastening portion for airtight connection to a connecting portion of a suction device, a suction opening and a fluid line from the fastening portion to the suction opening.

The present invention further relates to a kit for aspiration of body fluids, for example pus from pimples, comprising a suction device according to the invention and at least one suction attachment according to the invention,

Pimple suckers for aspirating purulent pimples and blackheads are known in the prior art. DE 202 04 268 U1 describes a pimple sucker in syringe shape, comprising a syringe body and a piston arranged in the syringe body in the form of a punch, wherein the punch handle is configured as a web or ring shape.

In DE 10 2008 003 762 B4 a pimple sucker is described, with a base body in the manner of a conventional syringe with piston sliding therein. This pimple sucker comprises a compression spring which presses the piston into its starting position. The piston is connected to a handle, which is pulled with the fingers for the purpose of suction against the ball of the thumb or the thumb. Two check valves, one arranged in the syringe jacket and another disposed between the syringe tip and a lip lens, provide for the generation of a vacuum during sucking in the lip lens. During the return stroke, the air can escape via the check valve from the syringe jacket.

The pimple suckers known from the prior art are impractical to handle and ineffective. For their handling, it is basically necessary to use both hands, which complicates self-treatment, especially at hard-to-reach places. In addition, the known pimple suckers have large dimensions which complicate their handling. With the known pimple suckers only a low vacuum can be reached, which is usually at −500 mbar and cannot be regulated.

Alternative techniques for treating for instance acne, for example ointments, require a long treatment time with unsafe success. The squeezing of pimples or blackheads by hand is also painful and can lead to scarring.

In view of the above-mentioned disadvantages, it is the object of the present invention to provide a manually actuated suction device for aspiration of body fluids that works reliably, has a compact structure and is easy to handle.

The present invention achieves this object in that in the above-mentioned manually actuated suction device for aspiration of body fluids, the negative-pressure chamber is arranged in stroke direction between the piston crown and a handling surface of the actuation device. In the known, syringe-like pimple suckers, the piston and the handle of the actuation device, with which the piston is moved, are always located on one side of the negative-pressure chamber. This has the consequence that the handle of the actuation device during the suction stroke moves away from the negative-pressure chamber, which inevitably leads to larger dimensions and difficult handling. In contrast, in the suction device according to the invention the negative-pressure chamber is arranged between the piston crown and the handling surface, which allows a more compact design and better handling of the suction device according to the invention.

The suction attachment according to the invention achieves the above object in that a liquid sorbent is arranged in the fluid line. In the pimple sucker of DE 10 2008 003 762 B4, the filter is integrated as a liquid sorbent in the pimple sucker. This makes the structure of the pimple sucker more complex and reduces its effectiveness, since the integration of a replaceable filter entails the risk of negative-pressure losses. According to the invention, the liquid sorbent is integrated in the suction attachment. As a result, the structure of the pimple suction device is simplified, and this prevents the contamination of components located between suction attachment and suction device, such as the connection line with check valve of DE 10 2008 003 762 B4.

The solutions according to the invention can be further improved by the designs described below, each of which is advantageous and can be combined with one another as desired.

According to an advantageous embodiment of the suction device, the aspiration opening may be arranged on a barrel bottom opposite to the piston crown. In this way, the vacuum generated in the negative-pressure chamber is available at the aspiration opening over the entire suction stroke.

The aspiration opening may, according to another embodiment, terminate in a suction nozzle. The suction nozzle may be made integral with the barrel. Alternatively, the suction nozzle can be arranged on the barrel as a separate component essentially vacuum-tightly surrounding the aspiration opening. The terms air-tight, vacuum-tight or pressure-tight are used in the following application as synonyms and mean that the device is impermeable to air at this place.

A compact design of the suction device according to the invention can be achieved by the suction nozzle extending substantially in stroke direction. A design in which the actuation direction along which the actuation device can be transferred from its starting position into its actuation position coincides with the stroke direction of the barrel/piston pair is also conducive to a compact construction of the suction device according to the invention.

According to a further embodiment, the distance between the negative-pressure chamber and the handling surface in the actuation position may be smaller than in the starting position of the actuation device. In this embodiment, the handling surface moves during actuation of the suction device towards the negative-pressure chamber, so that upon actuation of the suction device according to the invention, in contrast to the suction devices known from the prior art, its spatial dimensions are reduced, which facilitates handling significantly. According to a further embodiment, the distance between the piston crown and the handling surface in the actuation position may be smaller than in the starting position. This embodiment also allows a particularly compact design.

For the purposes of the present invention, the transfer of the actuation device from the starting position into the actuation position is referred to as actuation. Return designates the transfer of the actuation device from its actuation position into the starting position. Actuation and return together result in a pump or actuation cycle.

The distance between the negative-pressure chamber and the handling surface may be smaller than in the starting position especially in embodiments in which the actuation device acts directly on the piston. This may be the case, for example, in designs with fixed barrel and movable piston. In the case of an embodiment in which the distance between the piston crown and the handling surface is smaller in the actuation position than in the starting position, the actuation device can act directly on the barrel, which is the case, for example, with suction devices with a fixed piston and a barrel movable thereto. According to the invention designs are also covered where the distance between the negative-pressure chamber and the handling surface and the distance between the piston crown and the handling surface in the actuation position is smaller than in the starting position.

According to a further embodiment, the actuation device may be movably mounted relative to a stationary base body of the suction device. The actuation device may be movably mounted, for example, in stroke direction relative to the stationary base body, so that the actuation direction coincides with the stroke direction. According to the invention the actuation direction indicates along which direction the actuation device is moved from its starting position into its actuation position. The stroke direction indicates in which direction piston and barrel of a piston/barrel pair, which form a negative-pressure chamber, are displaceable relative to each other.

According to a further embodiment, the base body and the actuation device can form a suction device housing which encloses a suction device interior. In this way, the actuation device is designed in the form of a housing part, which facilitates the handling of the suction device according to the invention and reduces the number of required components and thus the complexity of the suction device.

According to a further embodiment, the volume of the suction device interior in the actuation position may be smaller than in the starting position. This facilitates the handling of the suction device according to the invention, since during actuation the volume of the suction device interior is reduced, which corresponds to a squeezing by hand as a manual actuation and is therefore ergonomically particularly favorable.

In a further embodiment, the suction nozzle can protrude, at least in sections, from the suction device housing. The suction nozzle can protrude out of the suction device housing, in particular in the actuation position, but alternatively both in the starting position and in the actuation position out of the suction device housing. In one embodiment, the suction nozzle may protrude from the actuation device. For example, the suction nozzle may be connected to the base body, or monolithically formed with the base body and protrude from the actuation device in sections. According to the invention, the end of the suction device or suction device housing from which the suction nozzle protrudes, or where the aspiration opening is located, is referred to as the suction-side end.

In order to simplify the manual actuation of the suction device, the suction device according to the invention may further comprise a guide which directs the transfer of the actuation device in actuation direction. According to one embodiment, the suction nozzle may form a guide element for the actuation device. For example, upon actuation, the actuation device can slide along an outer wall of the suction nozzle, so that the orientation of the suction nozzle predetermines the actuation direction. For example, the actuation direction may have an opening through which the suction nozzle extends.

in a further embodiment, the barrel may form a guide element for the actuation device. For example, the actuation device can be made cylindrical or barrel-shaped. In the case of a cylindrical embodiment of the actuation device, this device can be arranged to receive the barrel of the negative-pressure chamber, so that the barrel jacket of the actuation device and the barrel jacket of the barrel of the negative-pressure chamber slide along each other during actuation and thus predetermine the actuation direction.

In the case of a cylindrical configuration of the actuation device, the barrel bottom can form the handling surface of the actuation device. According to the invention, the term barrel is not limited to circular barrels, but stands for a body which is delimited by its jacket surface and at least one of its sectional surfaces (end faces). The sectional surface may be circular (circular barrel), but also oval, ellipsoid, a polygon or freeform.

According to a further embodiment, the base body can form a guide element for the actuation device. For example, a gap between the base body and barrel of the negative-pressure chamber may be formed, wherein this gap forms a guide element for the actuation device. In this embodiment, the actuation device can be mounted displaceably back and forth and guided in the gap.

In a further embodiment, the actuation device can sink in sections into the base body when actuated. This can be realized, for example, in that the actuation device is at least partially received in the base body upon actuation. In an exemplary embodiment, the actuation device can enter in sections into the gap between base body and barrel when actuated. This design has the advantage that the gap defines the actuation direction and thus directs the transfer of the actuation device into the actuation direction. As the actuation device sinks into the base body when actuated, it enters the gap, the suction device volume decreases, which simplifies the handling of the suction device.

In another embodiment, the actuation device may comprise the handling surface at one end and may be connected to the piston at the opposite end. The handling surface may be arranged at the suction-side end.

In a further embodiment, the piston may be cylindrical. In this case, for example, the jacket of the cylindrical piston may be located outside the negative-pressure chamber and connected to the actuation device. In this design, an end face of the cylindrical piston forms the piston or piston crown which is slidably received in the barrel of the negative-pressure chamber.

According to a further embodiment, the suction device can comprise at least one further negative-pressure chamber, which is formed by a barrel and a piston, which is accommodated in the barrel of the further negative-pressure chamber displaceably and with play, the piston or the piston crown closing the further negative-pressure chamber in a substantially airtight manner. In a suction device with at least two negative-pressure chambers, the required negative pressures of at least −750 mbar, preferably at least −850 mbar, can be achieved more quickly.

According to one embodiment, the barrel, which forms the (first) negative-pressure chamber, can also form the at least one further negative-pressure chamber. In another embodiment, the cylindrical piston of the (first) negative-pressure chamber can simultaneously form the barrel of the at least one further negative-pressure chamber, which represents a structurally compact solution, since the barrel of the (first) negative-pressure chamber and the barrel of the further negative-pressure chamber are of an interleaved, telescopic construction.

According to a further embodiment, a compensating element for pressure compensation may be provided between the (first) negative-pressure chamber and the further negative-pressure chamber. Such a compensating element may be formed, for example, as an overflow. The overflow may be released and allow pressure compensation as soon as the actuation device assumes a certain position, for example, its starting position or its actuation position. The overflow can also be enabled permanently. According to one embodiment, the inner wall of the barrel jacket may have a recess, for example a projection or circumferential groove, as a pressure compensation element. This embodiment can be used, for example, in suction devices with a barrel which forms two negative-pressure chambers which are separated from one another by a piston displaceably mounted in the barrel. If the point where the piston is sealed in an airtight manner relative to the barrel jacket impinges on the recess, pressure compensation takes place between the two negative-pressure chambers on the opposite sides of the piston.

A structurally particularly simple embodiment provides that the jacket of a cylindrical actuation device simultaneously forms the jacket of the barrel of the negative-pressure chamber and optionally also a further negative-pressure chamber.

The number of the movable components required for the suction device can alternatively or additionally be reduced by the fact that the piston or the barrel is fixedly connected to the base body. For example, the piston or the barrel may be formed integrally with the base body.

In order to maintain the negative pressure generated in the negative-pressure chamber, a valve may be provided that can be transferred into the closed state during a suction stroke and into the open state during an ejection stroke. Such a valve is also referred to below as outlet valve. Such a valve may be designed, for example, as a check valve. Self-medium actuated flat diaphragm valves are particularly space-saving. In such valves, a diaphragm, such as a movable flat rubber diaphragm, is contained, which depending on the pressure or pressure change of the surrounding medium opens or closes the valve. According to the invention, a suction stroke (also called pre-stroke) refers to the stroke operation in which the volume of the negative-pressure chamber rises, for example during extension of the piston, wherein a negative pressure is formed in the case of an airtightly dosed negative-pressure chamber. An ejection stroke, also called return stroke, characterizes the reverse stroke process, in which the volume of the negative-pressure chamber decreases, for example, the piston is retracted.

In a further embodiment, the piston may have a passage which is closed by an outlet valve, whereby the valve can be installed in a space-saving manner.

In a further embodiment, a second valve may be provided, which can be transferred into the open state during a suction stroke and can be transferred into the closed state during an ejection stroke. Such a second valve may preferably be arranged in the aspiration opening. Hereinafter, such a second valve is also referred to as a suction valve, since it ensures that the negative pressure built up in the negative-pressure chamber is passed on via the aspiration opening at the predetermined time. In designs with at least one further negative-pressure chamber, a further valve may be arranged between two adjacent negative-pressure chambers, which valve regulates the pressure compensation between the adjacent negative-pressure chambers. All valves of the suction device according to the invention can be designed as self-medium actuated flat diaphragm valves, which is structurally simple and ensures the opening or closing of the valves reliably and without additional effort and almost without any wear.

In order to facilitate a pumping cycle or repeated pumping cycles of the suction device according to the invention, a resetting device can be provided, by means of which a resetting force counter to the actuation direction can be generated, i.e. the resetting force acts in the direction along which the actuation device is transferred from the actuation position into the starting position, i.e. in resetting direction. For example, the resetting device may comprise a spring element. In one embodiment, the resetting device can move the actuation element into its starting position. Due to the fact that the resetting device presses the actuation element into its starting position, the actuation device must only be transferred manually by the user from its starting position into the actuation position. The resetting and the completion of the actuation cycle are subsequently carried out by the resetting device.

In a further embodiment, the suction nozzle may extend through the resetting device, which is particularly space-saving. In this design, the resetting device is mounted on the suction nozzle.

According to a further embodiment, the suction nozzle may comprise a connecting portion for airtight connection of a suction attachment. The connecting portion may for example have a seat on which the suction nozzle, preferably with the fastening portion of the attachment body, is connectable in a frictional and airtight manner. In the connecting portion or fastening portion, complementary connecting means can be used, for example, a screw connection, a bayonet lock or a clasp lock.

The suction device according to the invention can, in a further embodiment, comprise a cap which can be repeatedly attached to and detached from the suction device. The cap can enclose the suction nozzle and/or the handling surface in the attached state. The cap can thus unite in the attached state with the suction device housing to form a cubage.

In a further embodiment, the cap may comprise a fastening portion, which is configured to be connectable to the connecting portion of the suction nozzle. The fastening portion may be arranged in the interior of the cap. The interior of the cap is understood to be the space which encloses the cap.

In a further embodiment, the cap may comprise at least one carrier element for a suction attachment, so that the cap assumes a double function and can be used at the same time as a carrier body or bearing for a suction attachment. For example, the carrier element may be arranged in the interior of the cap, which has the advantage that the suction attachment can be arranged in the interior of the cap and can thus be stored protected by the cap against external influences.

In the following, exemplary designs of the suction attachment according to the invention will be discussed. It also applies here that the solution according to the invention of the suction attachment according to the invention can be improved by the following, mutually independent, respectively advantageous and arbitrarily combinable developments.

According to one embodiment of the suction attachment according to the invention, this attachment comprises a support surface, which is arranged in the fluid line at a predetermined distance from and along a defined contour relative to the suction opening. The support surface defines how far and in which form the skin is sucked through the suction opening into the suction attachment during treatment. In this way, tissue damage is prevented, which can occur when the skin is inadvertently sucked too far. Basically, the suction opening of the suction attachment relates according to the invention to the area on which, when the suction attachment is used, the negative pressure generated by the suction device acts. In this way, tissue damage can be prevented because the extent to which a tissue is drawn into the suction attachment is determined by the support surface.

According to a further embodiment, the suction attachment may comprise a support body which provides the support surface. The support body may be arranged, for example, in the fluid line. To prevent the support body from closing the fluid line, which closing should be avoided so that the vacuum generated with the suction device can act on the suction opening, the support body may comprise passage channels or consist of an air-permeable porous material.

According to a further embodiment, the suction attachment may comprise a scratching tool. By means of this scratching tool, the tissue can be perforated to improve fluid exit, for example, an acne pimple can be pierced. The scratching tool may be formed, for example, as a lancet, needle, pin, knife or point. A design of the scratching tool as a needle plate with at least two needles is also possible. The scratching tool can be made for example of glass, plastic or a metal.

In one embodiment, the scratching tool may be arranged on the support surface. As the tissue is sucked into the suction attachment through the suction opening and comes to rest on the support surface, the scratching tool can automatically perforate the tissue.

According to a further embodiment, the support surface can be arranged between the sorbent, for example a secretion filter, and the suction opening. Alternatively or additionally, the scratching tool can be arranged between the sorbent and the suction opening. In this way, the liquid sorbent, for example a liquid-receiving filter, is protected by the support surface and/or the scratching tool against contact by the tissue to be treated. At the same time, the support surface or the support body providing it and/or the scratching tool can hold the liquid sorbent in the fluid line.

According to a further embodiment, the suction attachment according to the invention may comprise a contour insert. The contour attachment has a contour, for example a cross, plus sign, or any outline, similar to a cookie cutter, which makes it possible to twist or to squeeze the pimple to be treated.

In order to ensure the correct handling of the suction attachment, this attachment, according to another embodiment, can comprise a magnifying lens, i.e. a magnifying glass. A magnifying lens makes it easier to place the suction attachment with its suction opening exactly at the site where the body fluid is to be aspirated. Also, the exemplary handling of a scratching tool, which will be explained in more detail hereinafter, is facilitated by a magnifying lens.

According to a further embodiment, the suction attachment may comprise a suction funnel that forms the suction opening. The suction funnel may be formed integrally with the suction opening at the suction-side end of the attachment body. The formation of a suction funnel has the advantage that one can increase the suction opening compared to the cross-sectional area of the fluid line or the fastening carriage of the attachment body and adapt the outline of the suction opening and also the contour of the adjoining interior of the fluid line, into which the tissue to be treated is sucked, specifically to the requirements of the suction attachment, e.g. as a cupping glass.

According to a further embodiment, the region near the suction opening, such as the suction funnel, can be transparent at least in sections, which facilitates the application of the suction attachment.

According to a further embodiment, a contact surface can surround the suction opening. The contact surface may for example have the shape of a sealing lip and ensures that an airtight seal on the boundary surface between the tissue to be treated and the suction attachment, i.e. the suction opening, takes place. Depending on the field of application, the suction opening may be oval, round, polygonal or free-form.

According to a further embodiment, the scratching tool can be resiliently mounted. This can prevent the tissue sucked through the suction opening from being pressed too strongly and painfully against the scratching tool. A resilient mounting limits the counter-pressure exerted by the scratching tool on the tissue to a desired level. In the same way, the support surface or the support body can be resiliently mounted.

According to a further embodiment, the scratching tool may be movably mounted relative to the suction opening. The scratching tool can, for example, be mounted so as to be rotatable relative to the suction opening or to be displaceable towards the suction opening or away from the suction opening. A movable mounting of the scratching tool allows its activation. For example, before or while negative pressure is being built up by the suction device, the scratching tool can be moved towards the suction opening in order to weaken or scratch the tissue at the desired location.

According to one embodiment, the attachment body may be constructed in two parts. In this case, a first part of the attachment body can be mounted movably, for example rotatably and/or displaceably, relative to the second part of the attachment body. The scratching tool can be arranged in a stationary manner on the first part of the attachment body. Due to the relative mobility of the first to the second part of the attachment body, the scratching tool can be moved together with the first part in the desired manner. Preferably, the second stationary part comprises the suction opening, so that the second part with suction opening can be placed on the desired location and the first part with the scratching tool is subsequently moved in the desired manner to the second part.

A design in which the attachment body is constructed in two parts makes it moreover easier to arrange the liquid sorbent and/or a support surface or a support body providing this support surface in the fluid line. For example, liquid sorbent and/or support surface or support body can be held between the first and the second part of the attachment body.

According to a further embodiment, the first part and the second part of the attachment body can be connected to one another to be rotatable and/or displaceable, in particular linearly displaceable with each other. A displaceable bond can be provided, for example, by providing a coupling element between the first and the second part of the attachment body, by means of which the distance between the first and the second part can be adjusted. For example, the first and second part of the attachment body may be connected together by a bellows that functions like an accordion and allows the first and second part of the attachment body to move toward and away from each other.

According to a further embodiment, the first and the second part of the attachment body can be connected to one another via a screw thread. A screw thread accomplishes that the first part of the attachment body is rotatable and displaceable relative to the second part of the attachment body.

According to a further embodiment, the suction attachment according to the invention can be packed in a sterile manner. A sterile packaging means any packaging in which the fluid line is sealed in an airtight manner both at the suction opening and at the fastening portion. This can be done, for example, by accommodating the suction attachment as a whole in an airtight packaging and then sterilizing it. It is also conceivable that only the suction opening and the fastening portion are sealed in an airtight manner and the suction attachment is then sterilized.

According to a further embodiment, the suction attachment can be designed as a disposable attachment which is to be disposed of after its use. It is also possible to design the suction attachment as a reusable attachment, which is cleaned after its use. Disposable/reusable combinations are also conceivable in which consumable material, such as the liquid sorbent or a support surface enclosing the suction opening or a suction funnel forming the suction opening, are exchanged after use, while the rest of the attachment body can be reused after cleaning and sterilization.

According to a further embodiment, the suction device according to the invention and/or the suction attachment according to the invention may comprise a vent. By means of a vent, the fluid line or the suction nozzle or the negative-pressure chamber of the suction device can be acted upon by ambient pressure to reduce the negative pressure. Instead of or in addition to a vent, the suction device according to the invention or the suction attachment according to the invention may comprise a pressure regulator. Such a pressure regulator ensures that an excessively high negative pressure is avoided. The pressure regulator may comprise a valve which automatically opens when a predetermined negative pressure is exceeded, and regulates the generated pressure or below the predetermined maximum negative pressure.

According to a further embodiment, the suction device according to the invention and/or the suction attachment according to the invention may comprise a pressure indicator which indicates the pressure prevailing in the fluid line or the suction nozzle and/or the negative-pressure chamber.

The present invention further comprises a kit for aspiration of body fluids, such as pus from pimples, which comprises a suction device according to the invention and at least one suction attachment according to the invention. The kit may further include a flexible connection hose having a fastening portion for airtight connection to the suction device at one end thereof, and a connecting portion for airtight connection of the suction attachment at the other end thereof.

The fastening portion of the connection hose may be configured to be connectable in an airtight manner to the connecting portion of the suction nozzle. The connecting portion of the connection hose may be designed to be connectable in an airtight manner to the fastening portion of the suction attachment. The airtight connection can be realized by analogy with the above-described airtight connection between suction opening or suction nozzle of the suction device and suction attachment according to the invention. A flexible connection hose facilitates handling, especially in hard-to-reach areas of the body, since the suction attachment is not attached directly to the suction device, but only the suction attachment fastened to the flexible connection hose is guided to the application site, whereas in comparison to the suction attachment significantly larger suction devices can be actuated at a place remote from the application site.

In the following, the invention will be explained in more detail by way of example with reference to embodiments with reference to the drawings. For the sake of simplicity, the same reference numerals are used in the figures for elements that correspond to one another in function and/or structure. It should also be noted that in accordance with the individual designs described above, individual elements of the embodiments can be omitted or added, depending on whether or not they are necessary for the particular application.

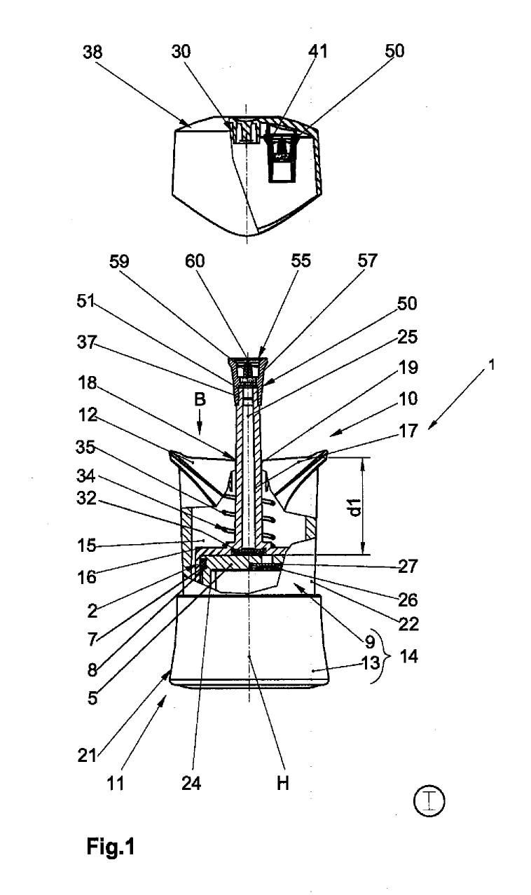

FIG. 1 shows a schematic, partially sectioned side view of a suction device according to the invention of a first embodiment with mounted suction attachment in its starting position;

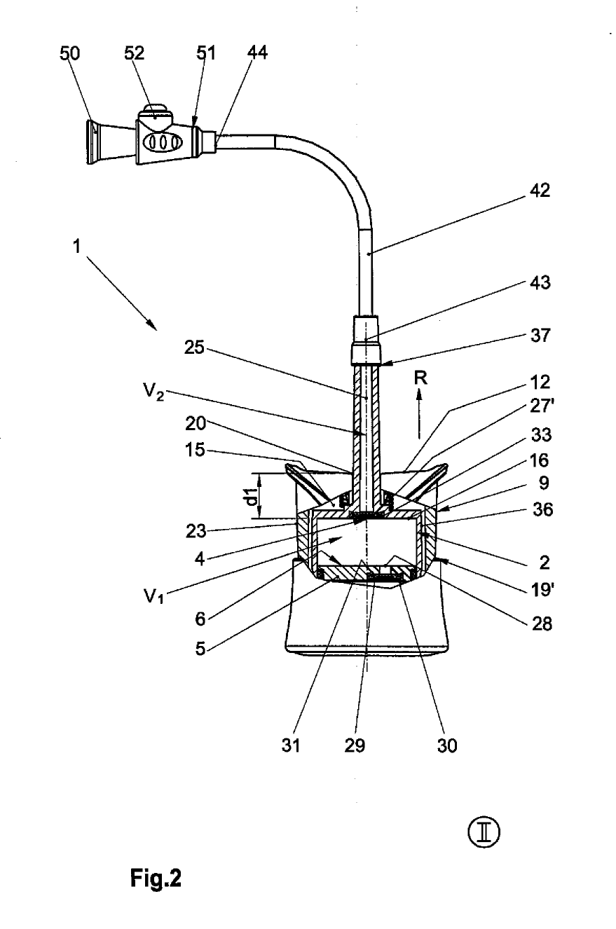

FIG. 2 shows a schematic, partially sectioned side view of the suction device of the embodiment of FIG. 1 in the actuation position, instead of a directly mounted suction attachment with a suction attachment in a further embodiment, which is connected via a flexible hose to the suction device;

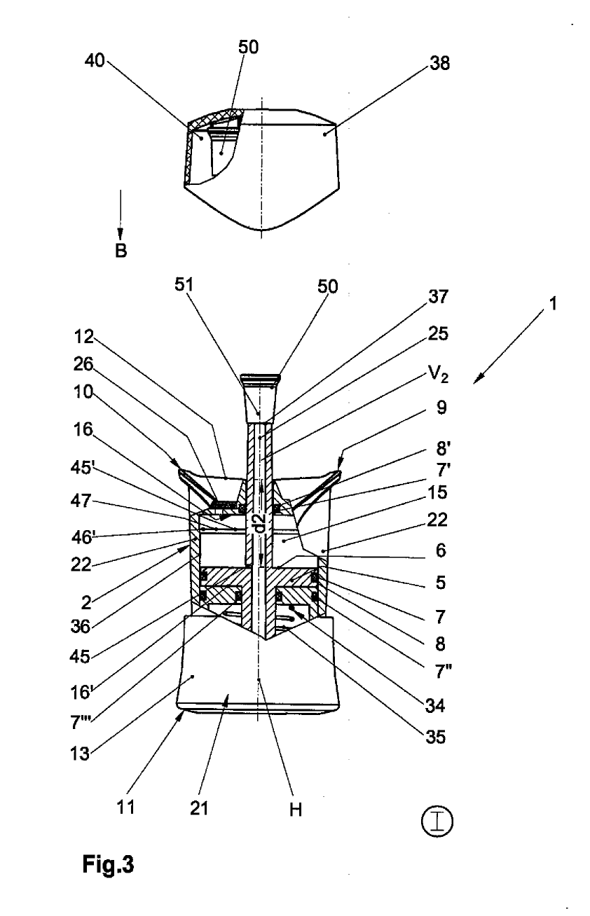

FIG. 3 shows a schematic, partially sectioned side view of a suction device according to the invention of an exemplary second embodiment with mounted suction attachment in the starting position;

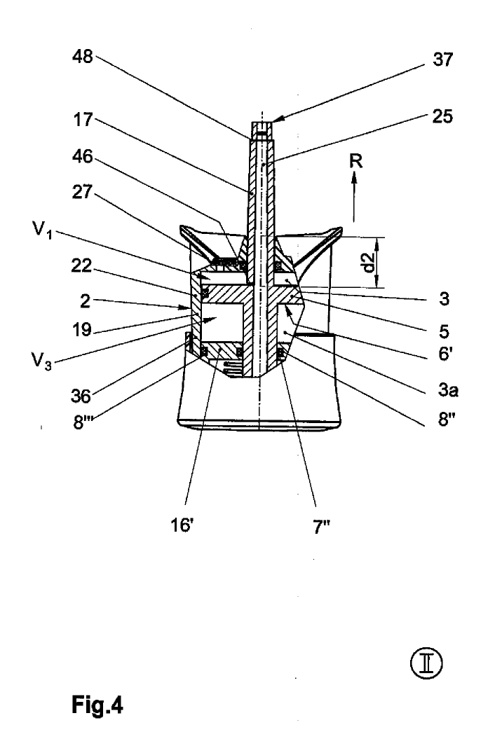

FIG. 4 shows the suction device of FIG. 3, in a schematic, partially sectioned side view in its operating position, without cap and without suction attachment;

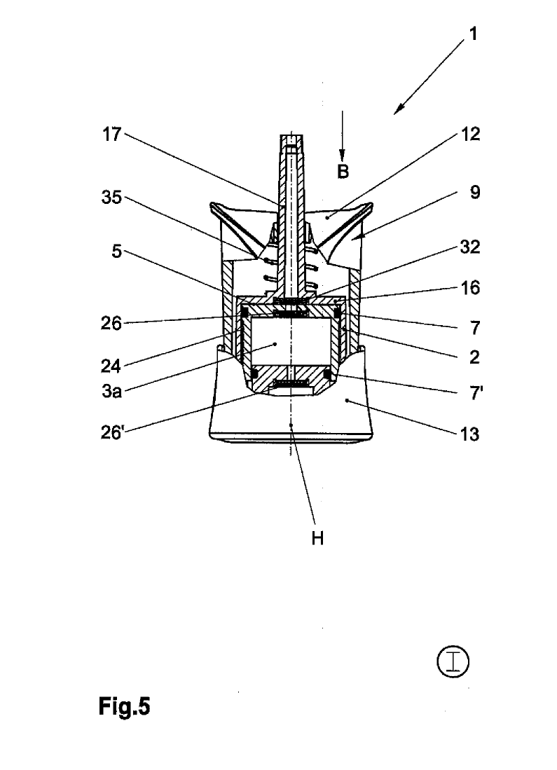

FIG. 5 shows a suction device according to the invention according to a third embodiment in a schematic, partially sectioned side view in its starting position;

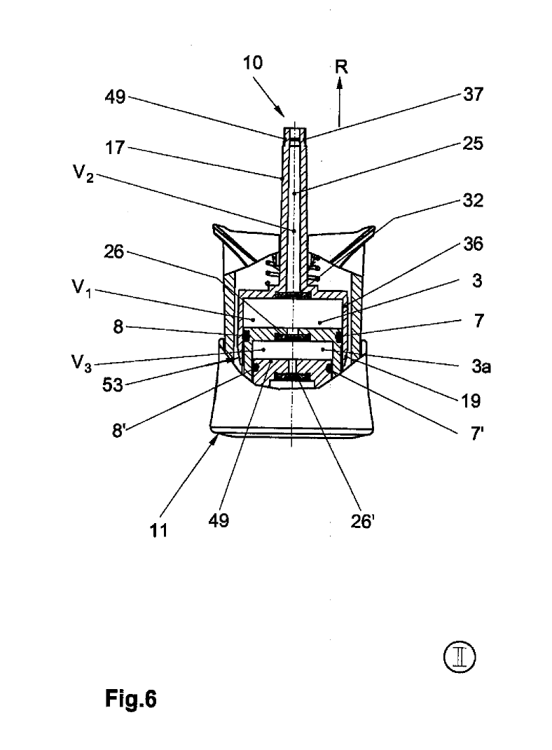

FIG. 6 shows a suction device according to the invention according to a third embodiment in the operating position;

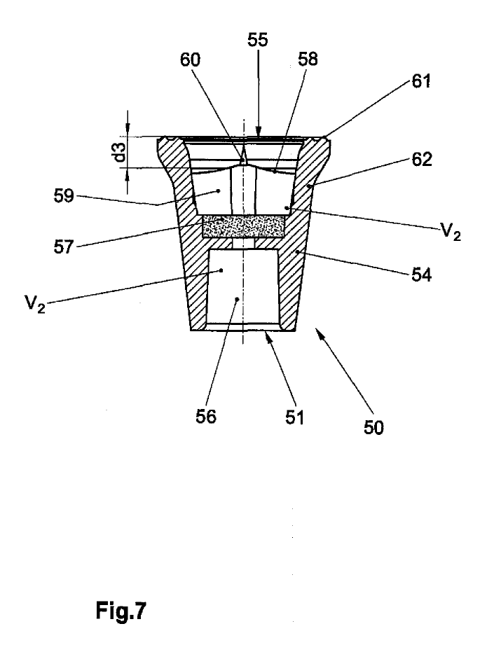

FIG. 7 shows a schematic sectional side view of a suction attachment according to the invention according to a second embodiment;

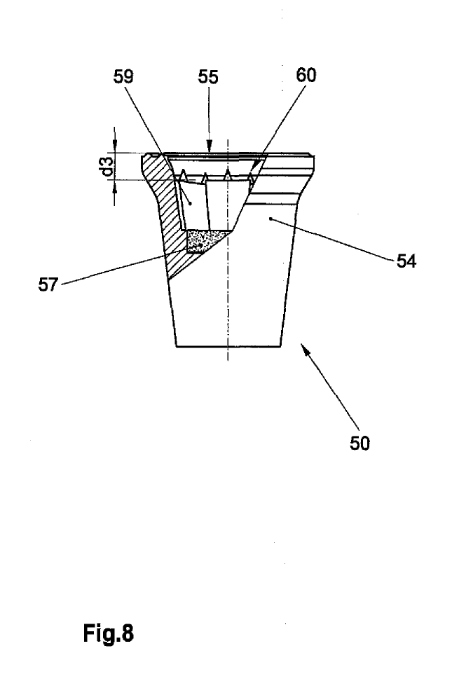

FIG. 8 shows a schematic, partially sectioned side view of a suction attachment according to the invention according to a third embodiment;

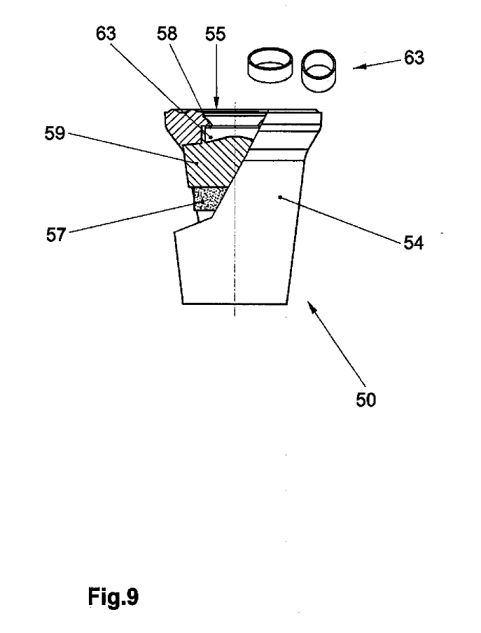

FIG. 9 shows a schematic, partially sectioned side view of a suction attachment according to the invention according to a fourth embodiment;

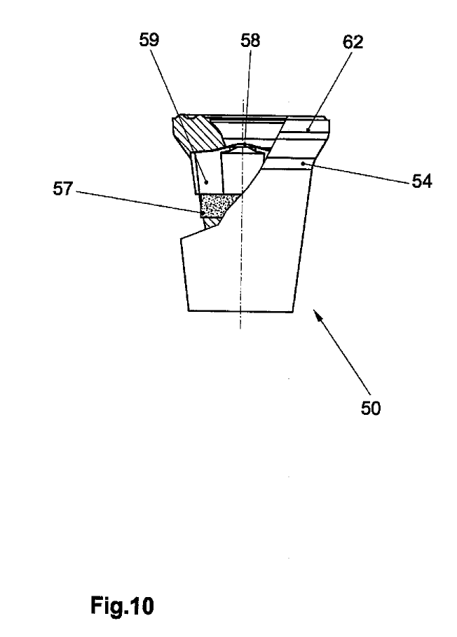

FIG. 10 shows a schematic, partially sectioned side view of a suction attachment according to the invention according to a fifth embodiment;

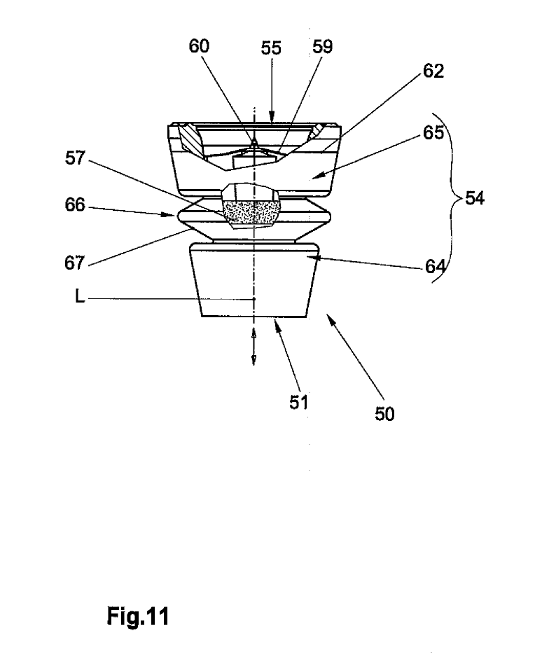

FIG. 11 shows a schematic, partially sectioned side view of a suction attachment according to the invention according to a sixth embodiment;

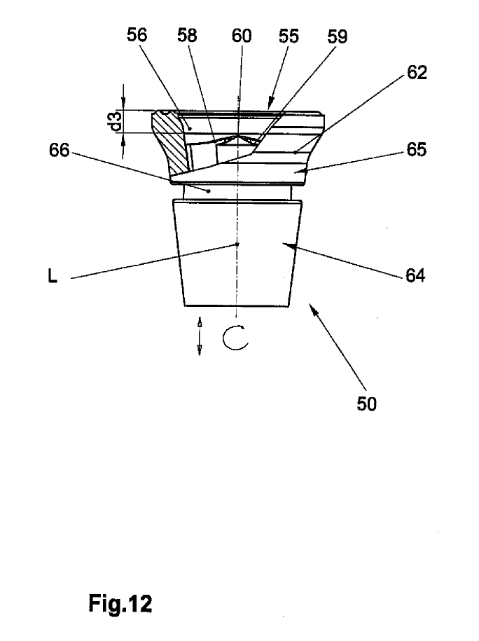

FIG. 12 shows a schematic, partially sectioned side view of a suction attachment according to the invention according to a seventh embodiment;

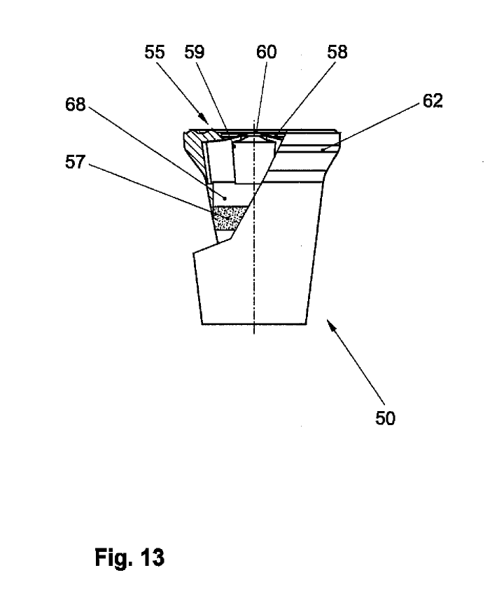

FIG. 13 shows a schematic, partially sectioned side view of a suction attachment according to the invention according to an eighth embodiment;

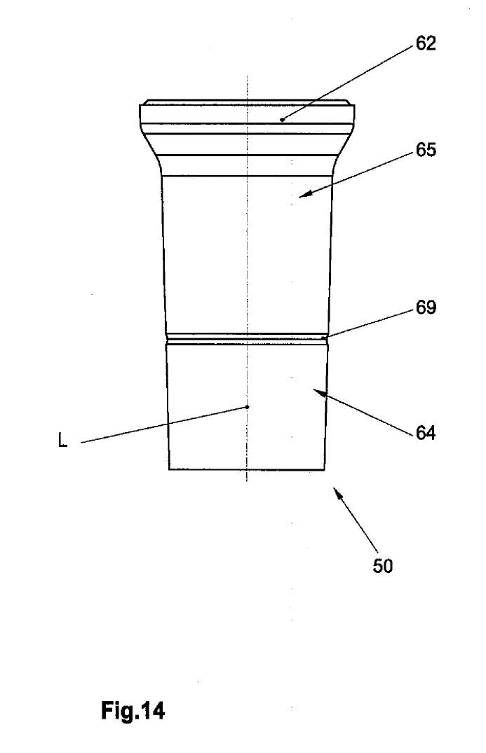

FIG. 14 shows a schematic, partially sectioned side view of a suction attachment according to the invention according to a ninth embodiment;

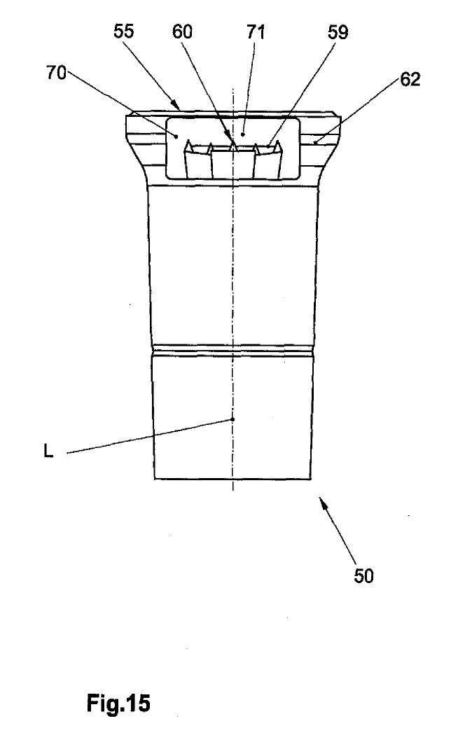

FIG. 15 shows a schematic, partially sectioned side view of a suction attachment according to the invention according to a tenth embodiment.

In the following, a first exemplary embodiment of a suction device 1 according to the invention, which is shown in FIGS. 1 and 2, is explained in more detail:

The manually actuated suction device 1 according to the invention for aspiration of body fluids, for example purulent pimples, nasal secretion, comprises a barrel 2 with a negative-pressure chamber 3, which comprises an aspiration opening 4. The suction device 1 further comprises a piston 5 which is slidably received in the barrel 2. The piston 5 is slidably received in the barrel 2 with play, its piston crown 6 closing the negative-pressure chamber 3 of the barrel 2 in a substantially airtight manner, so that when performing a suction stroke in the barrel chamber 3, a vacuum V₁ is generated, which will still be discussed in more detail below. For airtight sealing of the piston 5 relative to the barrel 2, a sealing ring 7 is provided in the embodiment shown. The sealing ring 7 is designed as a circumferential sealing ring, which is arranged in a radial sealing seat 8 of the piston. In the embodiment shown, the sealing seat 8 is formed as a radially circumferential groove in the piston 5.

The manually actuatable suction device 1 further comprises an actuation device 9. Upon actuation the actuation device 9 is designed to be transferrable from a starting position I (which is shown in FIG. 1) along an actuation direction B into an operating position II, which is shown in FIG. 2. By the actuation device 9, the piston 5 and the barrel 2 are displaceable relative to each other in stroke direction H. In the illustrated embodiment, the stroke direction H corresponds to the longitudinal axis of the suction device 1. The actuation direction B is parallel to the stroke direction H and points from the suction-side end 10 of the suction device 1 in the direction of the holding-side end 11 opposite thereto.

The actuation device 9 comprises a handling surface 12, which is preferably designed ergonomically, which is gripped by hand to actuate the actuation device 9. According to the invention, the negative-pressure chamber 3 is arranged between the piston crown 6 and the handling surface 12 of the actuation device 9.

The suction device 1 shown in FIGS. 1 and 2 comprises a base body 13 which together with the actuation device 9 forms a suction device housing 14, which encloses a suction device interior 15. In the embodiment shown, the barrel 2 with negative-pressure chamber 3 and the piston 5 are arranged in the suction device interior 15. The aspiration opening 4 is arranged on a barrel bottom 16 opposite to the piston crown 6. The aspiration opening 4 terminates in a suction nozzle 17 which in the illustrated embodiment is formed, for example, in one piece with the barrel 2. The suction nozzle 17 extends substantially in stroke direction H, namely away from the aspiration opening 4 in the barrel bottom 16 against the actuation direction B, i.e. In resetting direction R. The suction nozzle 17 protrudes from the suction-side end 11 of the suction device housing 14, and is used in the illustrated embodiment as a guide 18, which directs the transfer of the actuation device 9 from its starting position I into its operating position II in actuation direction B or again back into the starting position I in resetting direction R.

The suction nozzle 17 thus forms a guide element 19 in the first embodiment of the suction device 1 according to the invention. It specifies the direction in which the actuation device 9 is movably mounted. This is exemplified in the illustrated embodiment by the fact that the handling surface 12 has a passage opening 20 which radially surrounds the suction nozzle 17, so that the actuation device 9 is movable relative to the stationary base body 13 only in stroke direction H. In addition to the suction nozzle 17, the suction device 1 in the illustrated embodiment comprises a further guide element 19′. The further guide element 19′ is formed on the base body 13. The base body 13 has at its holding-side end 11 a handle 21 which is formed substantially pot-shaped. The inside of the handle 21 serves as a further guide element 19′, on which the actuation device 9 slides along when it is transferred from its starting position I into its actuation position II and back.

For this purpose, the actuation device 9 in the embodiment shown in FIGS. 1 and 2 is also substantially pot-shaped or cylindrical, wherein the ergonomically designed handling surface 12 forms the bottom of the pot/cylinder, which points in resetting direction R, i.e. away from the base body 13. The jacket 22 of the actuation device 9 is complementary to the jacket of the base body 12 which forms the guide element 19′. When the suction device 1 is actuated, the outside 23 of the jacket 22 of the actuation device 9 slides along the guide element 19′, which is formed by the pot-shaped base body 13 through the inside of the jacket.

When the suction device 1 is transferred from its starting position I shown in FIG. 1 into the actuation position II shown in FIG. 2, the part of the jacket 22 of the actuation device 9 which is assigned to the base body 13 sinks into the pot-shaped base body 13. The volume of the suction interior 15 is thus smaller in the actuation position II than in the starting position I, which proves the compact design of the suction device 1 according to the invention. In the actuation position II, the distance d₁ between handling surface 12 and negative-pressure chamber 3 is smaller than in the starting position II.

In the first exemplary embodiment of the suction device 1 shown in FIGS. 1 and 2, the base body 13 is fixedly connected to the barrel 2. The barrel 2 can be monolithic, i.e. one-piece, with the base body 13. The unit can be produced, for example, as an injection molded part. It is also possible to connect the barrel 2 rigidly to the base body 13. The actuation device 9 is mounted to be movable relative to the base body 13 and thus relative to the barrel 2.

The actuation device 9 has the handling surface 12 at its suction-side end 10. At the opposite, holding-side end 11, the actuation device 9 is connected to the piston 5. In the illustrated embodiment, the piston 5 is formed integrally with the actuation device 9. This is exemplarily realized in the illustrated embodiment in that the piston 5 is also cylindrical. The piston skirt 24 extends away from the piston crown 6 in actuation direction B and is connected to the jacket 22 of the actuation device 9. A movement of the actuation device 9 in actuation direction B thereby also pulls the piston 5 in actuation direction B.

To generate a vacuum V₁ or V₂ in the negative-pressure chamber 3 and in the interior 25 of the suction nozzle 17, the exemplary suction device 1 in the first embodiment comprises a valve 26. This valve, also called outlet valve 26, is transferrable in a suction stroke (in which in the first embodiment of FIGS. 1 and 2 the piston 6 is extended in stroke direction H, more precisely in actuation direction B, and the volume of the negative-pressure chamber 3 increases) into the closed state and is transferrable in an ejection stroke in reverse direction (i.e. In which the piston 6 is again retracted in resetting direction R and the volume of the negative-pressure chamber 3 decreases) into the open state. The outlet valve 26 thus forms a check valve. In the illustrated embodiment, the valve 26 is a self-medium actuated flat diaphragm valve which comprises a diaphragm disc 27. The outlet valve 26 is arranged by way of example in the piston 5, more precisely in the piston crown 6. The piston crown 6 has a passage 28 which is widened at its end pointing in actuation direction B. In this widening 29 sits the diaphragm 27, which is held in the widening 29 via a fastening sleeve or a fastening ring 30. When the piston 5 is extended, a negative pressure/vacuum V₁, which presses the diaphragm disc 27 against the shoulder 31 at the location of the widening 29 and thus closes the passage 28 and the valve 26, is formed in the negative-pressure chamber 3. In reverse direction, i.e. In the ejection or return stroke, the flat diaphragm 27 is moved in actuation direction B and thus away from the shoulder 21 and releases the passage 28; the valve 26 is open.

The first embodiment of the suction device 1 according to the invention comprises a second valve, a suction valve 32. The suction valve 32 is disposed in the aspiration opening 4. The suction valve 32 can be transferred into the open state during a suction stroke and can be transferred into the closed state during an ejection stroke. The suction valve 32, like the outlet valve 26, is also constructed as a self-medium fastened flat diaphragm valve. Since outlet valve 26 and suction valve 32 are arranged on sides of the negative-pressure chamber 3 which are opposite to each other against stroke direction H, the diaphragm 27′ of the suction valve 32 is also moved during the suction stroke in the direction of negative-pressure chamber 3, thereby, however, away from its shoulder 33, whereby the suction valve 32 opens during the suction stroke. In an analogous manner, the suction valve 32 closes during the return stroke.

The exemplary suction device 1 of the first embodiment further comprises a resetting device 34. The resetting device 34 can be used to generate a resetting force acting in the resetting direction R and thus counter to the actuation direction B. In the exemplary embodiment, the resetting device 34 comprises a spring element 35. The spring element 35 is formed by way of example as a compression spring. The resetting device 34 in general, and the spring element 35 in particular, moves the actuation element 9 into its starting position I. For this purpose, the resetting device 34 is arranged between the actuation device 9 and the barrel bottom 16. Upon actuation of the actuation device 9, the spring element 35 is compressed as soon as a force is exerted in actuation direction B on the handling surface 12. Taking away the actuating force from the handling surface 12, the spring element 35 relaxes and pushes the actuation element 9 back into its starting position I. In the illustrated embodiment, the resetting element 35 is arranged around the suction nozzle 17. In other words, the suction nozzle 17 extends through the resetting device 35 in the illustrated embodiment.

The suction nozzle 17 comprises a connecting portion 37 for the airtight connection of a suction attachment 50 according to the invention, which is described in more detail in an exemplary manner in the further figures and shown in an improved manner. The suction device 1 according to the invention may comprise a cap 38. The cap 38 is repeatedly attachable to and detachable from the suction device 1 in the illustrated embodiment. In the attached state (not shown in the figures), the cap 38 surrounds the suction nozzle 17, especially its part protruding from the suction device housing 14 and the handling surface 12.

The cap 38 comprises a fastening portion 39 in its interior 40, which is configured to be connectable to the connecting portion 37 of the suction nozzle 17. The fastening portion 39 may for example be a projection which is complementary to the mating face or seat 48 of the suction nozzle 17 and is frictionally connectable thereto. The cap 38 further includes a carrier element 41 for a suction nozzle 50 in the illustrated embodiment. The carrier element 41 is likewise arranged in the interior 40 of the cap 38. The cap thus not only serves as a closure means for the parts protruding from the suction device housing 14, but also acts as a bearing for suction attachments for the suction device. 1

In FIG. 2, in contrast to FIG. 1, no suction attachment 50 is mounted directly on the free end of the suction nozzle 17. Also, the cap is omitted in FIG. 2. In FIG. 2, a flexible connection hose 42 is provided instead. The connection hose 42 comprises a fastening portion 43 for airtight connection with the suction device 1, in the specific example with the connecting portion 37 of the suction nozzle 17, at its one end. At the other end, the connection hose 42 comprises a connecting portion 44 for airtight connection to a suction attachment 50. The fastening portion 43 of the hose 42 is made airtight with the connecting portion 37 of the suction nozzle 17, and the connecting portion 44 of the connection hose 42 is made airtight with a fastening portion 51 of the suction attachment 50.

The suction attachment 50 of the embodiment shown in FIG. 2 has a vent 52. By means of the vent 52, a vacuum applied in the suction attachment 50 can be reduced and atmospheric pressure can be produced in the suction attachment 50 for detaching the suction attachment 50.

The operation of the exemplary suction device 1 according to the first embodiment, which is shown in FIGS. 1 and 2, will now be explained in more detail:

For treatment, the cap 38 is first removed in the suction device 1 according to the invention. Subsequently, a suction attachment 50, which is mounted in the interior 40 of the cap 38, is placed on the free end of the suction nozzle 17. In this case, the fastening portion 51 of the suction attachment 50 is connected in an airtight manner to the connecting portion 37 of the suction nozzle 17. This state corresponds to the starting position I shown in FIG. 1.

The suction device according to the invention can be gripped by hand, so that the handle 21 of the base body 13 rests against the ball of the thumb and the fingers enclose the handling surface 12. Subsequently, the suction attachment is placed on the tissue to be treated, such as a purulent pimple, and is pumped to generate a vacuum. As a rule, only a few, for example three to five actuation cycles, are required. With the suction device 1 according to the invention a negative pressure of more than −750 mbar, preferably more than −850 bar, can usually be generated within less than five, preferably less than three actuation cycles, both in the negative-pressure chamber 3 and in the interior 25 of the suction nozzle 17 and thus in the suction nozzle 50 itself.

During the transfer of the suction device 1 from the starting position I to the actuation position II, the actuation device 9 is displaced in actuation direction B relative to the base body 13 by the user closing his hand. The actuation device 9 moves here in sections into the base body 13. At the same time, a suction stroke is performed in which the piston 5 is extended relative to the stationary barrel 2 in actuation direction. During extension a vacuum V₁ is generated in the negative-pressure chamber 3. In this case, the suction valve 32 opens, whereas the outlet valve 26 is closed. Due to the opened suction valve 32, the vacuum V₁ generated in the negative-pressure chamber 3 also extends to the interior 25 of the suction nozzle 17 (vacuum V₂) and the suction attachment 50, which is mounted in an airtight manner.

When resetting the actuation device 9 from its actuation position II in resetting direction R into its starting position I, which is performed by the resetting force generated by the resetting device 34, the suction valve 32 closes, so that the vacuum V₂ in the suction nozzle 17 and the suction attachment 50 is maintained. At the same time, the outlet valve 26 opens so that the air can escape from the negative-pressure chamber 3 via the outlet valve 26 before the next pump cycle occurs. One advantage is here that, at the beginning of the first pump cycle, there is always a working vacuum V₂ in the interior 25 of the suction nozzle 17 and in the suction attachment 50.

The use shown by way of example in FIG. 2 with a flexible connection hose 42 facilitates the treatment of hard-to-reach body parts. One embodiment of the suction attachment 50, in which a vent 52 is provided, facilitates the release of the sterile suction attachment 50 and allows control of the working vacuum V₂ in the suction nozzle 50 such that an excessively high negative pressure can be reduced by means of the vent 52.

In the following, a second, exemplary embodiment of the suction device 1 according to the invention will be described with reference to FIGS. 3 and 4. Here, for the sake of simplicity, the same reference numerals are used for elements that correspond in terms of function and/or structural elements to the first embodiment of FIGS. 1 and 2.

The structure of the base body 13 and the actuation device 9, as well as their mounting and guidance relative to each other, corresponds in the second embodiment substantially to that of the first embodiment. Likewise, the cap 38 and also the suction nozzle 50 correspond in the second embodiment shown in FIG. 3 to those of the first embodiment shown in FIG. 1.

In the second embodiment, however, the piston 5 is fixedly connected to the base body 13. In contrast, the barrel 2 is mounted to be movable relative to the fixed piston 5. In the illustrated embodiment, the jacket 22 of the actuation device 9 simultaneously represents the barrel jacket 36 of the barrel 2 of the negative-pressure chamber 3. In the suction device 1 of the second embodiment, the barrel 2 thus forms a guide element 19 for the actuation device 9.

The barrel bottom 16 is formed on the suction-side end 10 of the actuation device 9 with the handling surface 12 and is moved in actuation direction B relative to the suction nozzle 17. Therefore, the barrel bottom 16 of the negative-pressure chamber 3 has to be sealed against the suction nozzle 17. For this purpose, a further sealing seat 8′ in which a further seal 7′ is seated is provided in the barrel bottom 16.

In contrast to the suction device 1 of the first embodiment, the suction device 1 of the second embodiment shown in FIGS. 3 and 4 comprises a further negative-pressure chamber 3a. The further negative-pressure chamber 3a is also radially bounded in stroke direction H by the barrel 2, more precisely by its barrel jacket 36. At its suction-side end 10, which faces counter to the actuation direction B, the further negative-pressure chamber 3a is bounded by the fixed piston 5, wherein its end face pointing towards the holding-side end 11 forms the piston crown 6′ of the further negative-pressure chamber 3a. At its holding-side end, which points in actuation direction B, the jacket 22 of the actuation device 9 is closed by a further barrel bottom 16′. This barrel bottom 16′ at the holding-side end 11 of the actuation device 9 is moved together with the actuation device 9 in stroke direction H. The holding-side barrel bottom 16′ is sealed both with respect to the suction nozzle 17 and with respect to the jacket 22 of the barrel 2 and the actuation device 9, respectively, by sealing rings 7″ and 7″′ arranged in corresponding sealing seats 8″ and 8″′. The suction nozzle 17 extends in the second embodiment substantially from the holding-side end 11 of the handle 21 of the base body 13 through the entire suction interior 15 and beyond, since it protrudes from the suction side end 10 of the actuation device 9.

The outlet valve 26 is associated with the negative-pressure chamber 3 and is seated in its barrel bottom 16. Furthermore, the suction device 1 in the second embodiment comprises a compensating element 45 for pressure compensation between the negative-pressure chamber 3 and the interior 25 of the suction nozzle 17. In the illustrated embodiment, the compensating element 45 is designed as an overflow 46, for example in the form of a through hole through the suction nozzle 17 in the suction device interior 50, more precisely the negative-pressure chamber 3. In the illustrated embodiment, the overflow 46 is formed in the negative-pressure chamber 3 in the region of the fixed piston 5. The compensating element 45 ensures that the vacuum V₁ in the negative-pressure chamber 3 always corresponds to the vacuum V₂ in the suction nozzle 17 and thus the suction attachment 50.

The suction device 1 according to the second embodiment comprises a second compensating element 45′. The further compensating element 45′ is also arranged in the negative-pressure chamber 3 and ensures pressure compensation between the negative-pressure chamber 3 and the further negative-pressure chamber 3a. For this purpose, in the barrel jacket 36 in the negative-pressure chamber 3, a recess 47 is formed, which forms an overflow channel 46′. The recess 47 is formed as a projection or circumferential groove at the location of the barrel jacket 36, in which the sealing ring 7 is located in the actuation position II of the suction device 1 according to the invention of the second embodiment, which actuation position II is shown in FIG. 2. By the projection 47 of the overflow channel 46′, the airtight play between barrel 2 and piston 5 is interrupted when the seal 7 coincides with the overflow channel 46′, so that pressure compensation between the first negative-pressure chamber 3 and the second negative-pressure chamber 3a can take place.

In contrast to the suction device 1 of the first embodiment, the resetting device 34 is arranged in the second embodiment between the handle 21 of the base body 13 and the barrel bottom 16′ at the holding-side end 11 of the actuation device 9. The resetting device 34 also includes, in the second embodiment, a spring element 35 that, when the actuation device 9 is moved in actuation direction B into its actuation position II, is tensioned between barrel bottom 16′ and handle 21 and generates a resetting force, which, as soon as the pressure in actuation direction B on the handling surface 12 is dropping, moves the actuation device 9 in resetting direction R back into its starting position I.

In FIG. 4 the suction nozzle 50 and the cap 38 of FIG. 3 have been omitted for illustrative purposes. In FIG. 4, the connecting portion 37 is exemplified at the suction-side end of the suction nozzle 17. In the embodiment shown, the connecting portion 37 has a shoulder 48, which forms a seat for a fastening portion 51 of the suction attachment 50.

The operation of the suction device 1 of the second embodiment according to the invention will be explained briefly below.

In principle, the handling is analogous to the suction device 1 of the first embodiment.

When the actuation device 9 is transferred from its starting position I into its actuation position II in actuation direction B, the outlet valve 26, which is likewise provided analogously to the valves 26, 32 of the first embodiment as a self-medium actuated valve, opens and the air from the negative-pressure chamber 3 is discharged through the outlet valve 26 from the negative-pressure chamber 3. At the same time, a vacuum V₃ is built up in the further negative-pressure chamber 3a.

In the actuation position II, the compensating element 46′, namely the projection 47, coincides with the sealing ring 7 between piston 5 and jacket 2 of the first negative-pressure chamber 3, so that pressure compensation takes place between the first negative-pressure chamber 3 and the second negative-pressure chamber 3a. In this case, the failure valve 26 closes and the vacuum V₁ is passed on through the overflow channel 46 also as a vacuum V₂ into the interior 25 of the suction nozzle. In case of a transfer over the actuation device 9 from its actuation position II in resetting direction R back into its starting position I, the outlet valve 26 remains closed and in the negative-pressure chamber 3, the vacuum V₁ (and consequently also the vacuum V₂) is further increased, whereas there is a reduction against the vacuum V₃ in the further negative-pressure chamber 3a. This pump cycle is repeated in principle, with the exception that in the negative-pressure chamber 3 a vacuum V₁ is always present, which keeps the outlet valve 26 always closed in the subsequent pump cycles. The outlet valve 26 thus opens only at the very first pump stroke of the first actuation cycle. Instead of the overflow 46, a suction valve 32 (not shown in FIGS. 3 and 4) may also be installed between the first negative-pressure chamber 3 and the interior 25 of the suction nozzle 17, which suction valve 32 is configured to be transferrable during the suction stroke into the open state and to be transferrable during the ejection stroke into the closed state.

In the following, an exemplary third embodiment of a suction device 1 according to the invention is explained in more detail with reference to FIGS. 5 and 6. The structure of the suction device 1 of the third embodiment substantially corresponds to the suction device 1 of the first embodiment of FIGS. 1 and 2. In the following, only the differences between the suction devices of the first and the third embodiment will be discussed in more detail.

In FIGS. 5 and 6, the cap 38 and the suction attachment 50 are omitted for the sake of simplicity. At the suction-side end 10 of the suction nozzle 17, an external thread 49 is provided as a connecting portion 37 of the nozzle 17, on which a suction cap 50 with its fastening portion 51, which then has an internal thread (not shown), can be connected in an airtight manner.

Also, the suction device 1 of the third embodiment comprises a second negative-pressure chamber 3a. The barrel of the further negative-pressure chamber 3a is formed by the cylindrical piston 5 with its piston skirt 24. A stationary second piston 49 which is connected to the base body 13 is arranged in this displaceable piston cylinder. The piston 49 has a structure analogous to the piston 5 with a corresponding sealing ring 7′ in a sealing seat 8′, so that the piston 49 is received with play in the cylindrical piston 5, which closes the further negative-pressure chamber 3a in a substantially airtight manner. The piston 49 also comprises an outlet valve 26′, which corresponds in its design and arrangement to the outlet valve 26 in the piston 5 of the first negative-pressure chamber 3.

The handling of the suction device 1 according to the third embodiment is analogous to the handling of the suction device 1 of the first embodiment. During the transfer of the suction device from the starting position I (FIG. 5) in actuation direction B into its actuation position II (FIG. 6), a vacuum V₁ is built up in a manner analogous to the suction device 1 of the first embodiment in the negative-pressure chamber 3. In this case, the suction valve 32 opens, the outlet valve 26 closes. In the negative-pressure chamber 3a, the further outlet valve 26′ in the piston 49 opens and the negative-pressure chamber 3a is emptied via the further outlet valve 26′. Due to the opened suction valve 32, the negative pressure V₁ in the negative-pressure chamber 3 corresponds to the vacuum V₂ in the interior 25 of the suction nozzle 17. During resetting in resetting direction R from the actuation position II into the starting position I the suction valve 32 closes, so that the vacuum V₂ applied to the suction nozzle or suction attachment is maintained. During resetting a vacuum V₃ is generated in the second negative-pressure chamber 3a. The outlet valve 26′ closes and the vacuum V1 in the first negative-pressure chamber 3 is reduced.

In the second and in each subsequent suction stroke, the negative pressure in the negative-pressure chamber 3 increases, so that the valve 26 opens and there is pressure compensation between negative pressure V₁ and vacuum V₃ in the negative-pressure chambers 3 and 3a. As the negative pressure in the chamber 3 is reduced during the return stroke, the valve 26 closes again, the increased vacuum V₁ remains in the negative-pressure chamber 3. In the event that the air movement for closing the valve 26 is not sufficient, the diaphragm 27 in the valve 26 could be biased into the closed position. For the bias, the diaphragm 27 may be provided with a sealing lip (not shown), which is held via fastening sleeve 30 and acts on the diaphragm 27 analogously to a spring pressure.

Due to the two-chamber system, a high vacuum of over −850 mbar can be generated quickly within a few movement cycles (usually not more than three to four cycles).

In the suction device 1 of the third embodiment, a gap 53, which is accessible in actuation direction B, is formed between the base body 13 and the barrel 2, more precisely its jacket 36. This gap 53 forms a guide element 19 for the actuation device 9 in the third embodiment of the suction device 1 according to the invention. During transfer of the actuation device 9 into the actuation position II, the actuation device 9 moves in sections into this gap 53.

In the following, various exemplary embodiments of the suction attachment 50 according to the invention will be explained in more detail with reference to FIGS. 7-15. In FIGS. 7-13, the embodiments of the suction attachment 50 according to the invention are shown in a side view, partially in section.

The suction attachment 50 for a suction device 1 comprises an attachment body 54 with a fastening portion 51 for airtight connection with the connecting portion 37 of the suction nozzle 17 of the suction device 1 or the connecting portion 44 of a flexible hose 42. The suction attachment 50 further comprises a suction opening 55 and a fluid line 56 from the fastening portion 51 to the suction opening 55. If the suction attachment 51 is properly connected to the suction device 1, the vacuum V₂ prevailing in the suction nozzle 17 extends through the fluid line 56 to the suction opening 55, which during treatment abuts in an airtight manner on the tissue to be treated, so that the vacuum V₂ is available for aspiration of body fluids.

In the fluid line 56, a liquid sorbent 57 is arranged, which receives the aspirated liquid and prevents it from entering the suction device 1. The liquid sorbent 57 can be designed, for example, as a secretion filter.

The suction attachment 50 shown by way of example in FIG. 7 comprises a support surface 58, which is arranged in the fluid line 56 at a predetermined distance d₃ and along a defined contour to the suction opening 55. The support surface 58 is provided by a support body 59. The support body is arranged in the fluid line 56 between liquid sorbent 57 and suction opening 55, wherein its surface pointing into suction opening 55 forms the support surface 58.

The suction attachment 50 of the embodiment shown in FIG. 7 further comprises a scratching tool 60. The scratching tool 60 is arranged on the support surface 58 and formed by way of example as a needle protruding from the support surface 58 in the direction of the suction opening 55. In the embodiment shown, the scratching tool 60 is made integral with the support body 59. In use, the tissue to be treated is aspirated through the suction opening 55 into the fluid line 56 due to the vacuum V₂ until it rests against the support surface 58. In this process the scratching tool 60 perforates the skin of the tissue, for example a purulent pimple, opens it and thus facilitates the aspiration of pus fluid.

The suction opening 55 is surrounded by a contact surface 61, which ensures an air-tight seal between suction attachment 50, especially its suction opening 55, and the tissue to be treated and at the same time defines the shape of the suction opening 55, which is for example, oval, round, polygonal or freeform, depending on the application.

In the embodiment shown in FIG. 7, the suction attachment 50 is provided with a suction funnel 62. In the illustrated embodiment, this suction funnel 62 forms the suction opening 55. The suction funnel 62 may, at least in sections, be transparent, which facilitates a correct placement of the suction attachment 50. The design with a suction funnel 62 also facilitates the insertion of the liquid sorbent 57 and/or the support body 59 into the fluid line 56. For example, as shown in FIG. 8, liquid sorbent 57 and support body 59 can be inserted through the suction opening of the funnel 62 into the fluid line 56 and held frictionally within the attachment body 54.

The suction attachment 50 in the embodiment shown in FIG. 8 essentially corresponds to the suction attachment of the embodiment shown in FIG. 7. In the embodiment shown in FIG. 8, however, the suction opening 55 is somewhat larger and instead of a single needle as a scratching tool 60, a needle plate with a plurality of needles is used in the embodiment shown in FIG. 8.

The exemplary suction attachment 50 shown in FIG. 9 has, instead of a support body 58, a contour attachment 63 which provides the support surface 58. The use of contoured bodies 63 has the advantage that depending on the selection of the corresponding contour insert 63 the support surface 58 can be varied in a simple manner.

The suction attachment shown by way of example in FIG. 10 essentially corresponds to the suction attachment from FIG. 7, with the exception that the scratching tool 60 is omitted therein.

The scratching tool 60 may be movably mounted relative to the suction opening 55. The scratching tool 60 can be mounted to be, for example, rotatable and/or displaceable relative to the suction opening 55.

In the embodiment shown in FIG. 11, the scratching tool 60 is displaceable along the longitudinal axis L of the suction attachment 50, as indicated by the arrows on the right. For this purpose, the attachment body 54 is constructed in two parts with a first part 64 and a second part 65. In the embodiment shown in FIG. 11, the second part comprises a suction funnel 62, which forms the suction opening 55. The first part 64 of the attachment body 54 comprises the fastening portion 51 and is fixedly connected to the support body 58, which comprises the scratching tool 60. The first part 64 of the attachment body 54 is movable relative to the second part 65. For this purpose, the first part 64 and the second part 65 are displaceably connected to each other. The displaceable connection is realized by a coupling 66. In the embodiment shown in FIG. 11, the coupling 66 is designed as a bellows 67, which can be extended or shortened in the manner of an accordion and thus allows a displacement of the first part 64 relative to the second part 65. The displaceability makes it possible to place the second part 65 of the attachment body 54 with its suction opening 55 on the desired tissue site. Subsequently, the first part 64, which is fixedly connected to the scratching tool 60, can be moved in the direction of the second part 65, whereby the scratching tool 60 approaches the suction opening 50 and in this way one can manually control the pressure of the scratching tool 60 on the tissue to be treated and adjust it to a desired degree.

The exemplary embodiment of a suction attachment 50 shown in FIG. 12 basically corresponds to the embodiment shown in FIG. 11. Instead of a bellows 67 as a coupling element 66, however, a coupling element 66 is provided in the embodiment shown in FIG. 12, which allows axial rotation of the first part 64 about the longitudinal axis L relative to the second part 65. For this purpose, for example, the second part 65 forming the suction funnel 62 may be provided on its inner wall with a circumferential groove into which a spring projecting from the outer jacket of the first part 64 engages, which spring can rotate in the groove. Such an axially rotatable coupling 66 allows a rotational movement of the parts 64, 65 of the attachment body 54 that are otherwise fixed in the longitudinal axis L relative to each other. The rotation of the first part 64 about the longitudinal axis L rotates the scratching tool 60, which thus can be actively moved relative to a tissue sucked through the suction opening 55 into the fluid line 56 and resting on the support surface 58. The scratching tool 60 can be designed, for example, as a knife, which incises the tissue by way of the rotational movement.

Instead of a tongue-and-groove coupling 66 which only allows a rotatable mounting about the longitudinal axis L, a screw thread 66 may also be provided as a coupling element 66 in the embodiment shown in FIG. 12. In this case, the first part 64 would be mounted to be axially rotatable about the longitudinal axis and slidable in the longitudinal axis direction L relative to the second part 65. Such a rotatable and axially displaceable mounting makes it possible to successively build up the pressure of the scratching tool 60, which is fixedly connected to the first part 64, since this tool can be displaced almost continuously in the direction of the suction opening 55. In such an embodiment, therefore, the distance D₃ between suction opening 55 and support surface 58 can be adjusted continuously.

The exemplary suction attachment 50 shown in FIG. 13 essentially corresponds in its construction to the suction attachment shown in FIG. 10.

In FIG. 13, however, the support body 59 is resiliently mounted. For this purpose, a spring element 68 is arranged at the end of the support body 59, which element points away from the suction opening 55. By means of the spring element 68, the maximum pressure exerted by the scratching tool 60 on a sucked tissue can be regulated, since the resiliently mounted scratching tool 60 recedes and its counter-pressure exerted by it can be adjusted via the spring element 68.

The suction attachment 50 according to the invention can be designed both as one part (e.g. FIGS. 7-9) and as several parts (e.g. FIG. 11 or 12). The suction attachment 50 can also be constructed both as a disposable part, which is disposed of after single use, and as a reusable element. An exemplary embodiment as a reusable suction attachment 50 is shown in FIG. 14.

In this case, the first part 64 and the second part 65 are joined in a vacuum-tight manner at a seam 69. The parts 64 and 65 may be made of a reusable material, such as metal, but also of glass or a high-quality plastic, which can be sterilized. A liquid sorbent (not shown in FIG. 14) or a support body 59 (not shown in FIG. 14) may be formed of either a reusable sterilizable material or replaceable disposable elements that can be exchanged after disassembly of the parts 64 and 65.

Also conceivable are combinations of a reusable base body with single-use attachments. For example, the second part 65 may include a suction funnel attachment 62 which is disposed of as a skin-contacting portion after use and replaced by a new, sterile suction funnel 62.

Finally, FIG. 15 shows a further embodiment of a suction attachment 50 according to the invention. The suction attachment 50 of FIG. 15 basically corresponds in its construction to the suction attachment 50 shown in FIG. 14. In the illustrated embodiment, the suction attachment 50 is provided with a support body 59, which has a needle plate as a scratching tool 60 with needles projecting in the direction of the suction opening 55. In the region of the suction funnel 62, the suction attachment 50 is provided with a transparent window 70. In the illustrated embodiment, the transparent window is equipped with a magnifying lens 71, which facilitates correct application of the suction attachment 50.

### REFERENCE NUMERALS

1 Suction device

2 barrel

3 negative-pressure chamber

3a further negative-pressure chamber

4 aspiration opening

5 piston

6, 6′ piston crown

7, 7′, 7″, 7′″ sealing ring

8, 8′, 8″, 8′″ sealing seat

9 actuation device

10 suction-side end

11 holding-side end

12 handling surface

13 base body

14 suction device housing

15 suction device interior

16, 16′ barrel bottom

17 suction nozzle

18 guide

19, 19′ guide element

20 passage opening

21 handle

22 jacket of the actuation device

23 outside of the jacket 22

24 piston skirt

25 interior of the suction nozzle

26, 26′ (outlet) valve

27, 27′ diaphragm

28 passage in the piston crown

29 widening in the passage 28

30 fastening sleeve

31 shoulder of the outlet valve

32 (suction) valve

33 shoulder of the suction valve

34 resetting device

35 spring element

36 barrel jacket

37 connecting portion of the nozzle

38 cap

39 connecting portion of the cap

40 interior of the cap

41 carrier element

42 connection hose

43 fastening portion of the hose

44 connecting portion of the hose

45, 45′ compensating element

46, 46′ overflow (channel)

47 recess/projection

48 shoulder/seat

49 second piston

50 suction attachment

51 fastening portion

52 vent

53 gap

54 attachment body

55 suction opening

56 fluid line

57 liquid sorbent

58 support surface

59 support body

60 scratching tool

61 contact surface

62 suction funnel

63 contour insert

64 first part of the attachment body

65 second part of the attachment body

66 coupling

67 bellows

68 spring element

69 seam

70 inspection window

71 magnifying lens

I starting position

II actuation position

B actuation direction

d₁ distance from negative-pressure chamber to handling surface

d₂ distance from piston crown to handling surface

d₃ distance from suction opening to support surface

H stroke direction

L longitudinal axis

R resetting direction

V₁ vacuum in negative-pressure chamber

V₂ vacuum in suction nozzle

V₃ vacuum in second negative-pressure chamber

1. A manually actuated suction device (1) for aspiration of body fluids, for example pus from pimples, comprising a barrel (2) with a negative-pressure chamber (3), which has an aspiration opening (4), a piston (5) which is displaceable in the barrel (2) and is received with play, a piston crown (6) closing the negative-pressure chamber (3) of the barrel (2) in a substantially airtight manner, and an actuation device (9) which is transferrable from a starting position (I) along an actuation direction (B) to an actuation position (II) and by which the piston (5) and the barrel (2) are displaceable relative to each other in stroke direction (H), wherein the negative-pressure chamber (3) is arranged in stroke direction (H) between the piston crown (6) and a handling surface (12) of the actuation device (9).

2. Suction device (1) according to claim 1, wherein the aspiration opening (4) terminates in a suction nozzle (17).

3. Suction device (1) according to claim 1, wherein the distance (d₁) between the negative-pressure chamber (3) and the handling surface (12) and/or the distance (d₂) between the piston crown (6) and the handling surface (12) in the actuation position (II) is smaller than in the starting position (I).

4. Suction device (1) according to claim 1, wherein the actuation device (9) is movably mounted relative to a stationary base body (3), and wherein the base body (3) and the actuation device (9) form a suction device housing (14) that encloses a suction device interior (15).

5. Suction device (1) according to claim 4, wherein the suction nozzle (17) protrudes from the suction device housing (14) in sections.

6. Suction device (1) according to claim 1, further comprising a guide (18) which directs the transfer of the actuation device (9) in actuation direction (B).

7. Suction device (1) according to claim 1, wherein the suction device (1) comprises at least one further negative-pressure chamber (3a), which is formed by a barrel (2, 24) and a piston (5, 49) which is received in the barrel (2, 24) with play, the piston (5, 49) closing the further negative-pressure chamber (3a) in a substantially airtight manner.

8. Suction device (1) according to claim 7, comprising a compensating element (45, 45′) for pressure compensation between the negative-pressure chamber (3) and the further negative-pressure chamber (3a).

9. Suction device (1) according to claim 1, further comprising an outlet valve (26, 26′), which is transferrable into the closed state during a suction stroke and into the open state during an ejection stroke.

10. Suction attachment (50) for a suction device (1) for aspiration of body fluids, such as pus from pimples, comprising an attachment body (54) with a fastening portion (51) for airtight connection to a suction device (1), a suction opening (55) and a fluid line (56) from the fastening portion (51) to the suction opening (55), wherein a liquid sorbent (57) is arranged in the fluid line (56).

11. Suction attachment (50) according to claim 10, further comprising a support surface (58) which is arranged in the fluid line (56) at a predetermined distance (D3) from and along a defined contour to the suction opening (55).

12. Suction attachment (50) according to claim 10, further comprising a scratching tool (60).

13. Suction attachment (50) according to claim 10, wherein the scratching tool (60) is movably mounted relative to the suction opening (55).

14. Suction attachment (50) according to claim 10, wherein the attachment body (54) is constructed in two parts, and wherein preferably a first part (64) of the attachment body (54) is movably mounted relative to the second part (65) of the attachment body (54).

15. Kit for aspiration of body fluids, such as pus from pimples, comprising a suction device according to claim 1 and at least one suction attachment comprising an attachment body (54) with a fastening portion (51) for airtight connection to a suction device (1), a suction opening (55) and a fluid line (56) from the fastening portion (51) to the suction opening (55), wherein a liquid sorbent (57) is arranged in the fluid line (56), and optionally a connection hose (42) with a fastening portion (43) for airtight connection to the suction device (1) at its one end and with a connecting portion (44) for airtight connection of the suction attachment (50) at its other end.

16. Suction device (1) according to claim 2, wherein the distance (d₁) between the negative-pressure chamber (3) and the handling surface (12) and/or the distance (d₂) between the piston crown (6) and the handling surface (12) in the actuation position (II) is smaller than in the starting position (I).

17. Suction device (1) according to claim 2, wherein the actuation device (9) is movably mounted relative to a stationary base body (3), and wherein the base body (3) and the actuation device (9) form a suction device housing (14) that encloses a suction device interior (15).

18. Suction attachment (50) according to claim 11, wherein the attachment body (54) is constructed in two parts, and wherein preferably a first part (64) of the attachment body (54) is movably mounted relative to the second part (65) of the attachment body (54).

19. Suction attachment (50) according to claim 12, wherein the attachment body (54) is constructed in two parts, and wherein preferably a first part (64) of the attachment body (54) is movably mounted relative to the second part (65) of the attachment body (54).

20. Suction attachment (50) according to claim 13, wherein the attachment body (54) is constructed in two parts, and wherein preferably a first part (64) of the attachment body (54) is movably mounted relative to the second part (65) of the attachment body (54).
